(19) United States
(12) Patent Application Publication
(10) Pub. No.: US 2023/0390740 A1
(43) Pub. Date: Dec. 7, 2023

(54) CATALYST FOR CATALYTIC CRACKING OF ETHYLENEDICHLORIDE TO VINYL CHLORIDE

(71) Applicant: SCG CHEMICALS CO., LTD., Bangkok (TH)

(72) Inventors: Montree Namkajorn, Bangkok (TH); Churat Tiyapiboonchaiya, Bangkok (TH); Atsadawuth Siangsai, Bangkok (TH)

(21) Appl. No.: 18/020,612

(22) PCT Filed: Aug. 11, 2021
(86) PCT No.: PCT/TH2021/000045

§ 371 Date: Feb. 9, 2023

(30) Foreign Application Priority Data
Aug. 11, 2020 (SG) 10202007662V

(51) Int. Cl. B01J 21/18; B01J 21/08; B01J 23/04; B01J 35/00; B01J 35/10; C07C 17/25

(52) U.S. Cl. CPC B01J 21/18; B01J 21/08; B01J 23/04; C07C 17/25; B01J 35/1057; B01J 35/1028; B01J 35/0066

(57) ABSTRACT

An N-doped activated-carbon catalyst composition for essentially metal-free catalytic conversion of ethylenedichloride (EDC) into vinyl chloride monomer (VCM).

### CROSS-REFERENCE TO RELATED APPLICATION

This application claims the benefit of priority of Singapore Patent Application No. 10202007662V, filed 11 Aug. 2020, the content of it being hereby incorporated by reference in its entirety for all purposes.

### TECHNICAL FIELD

The present invention relates to catalyst for catalytic cracking of vinyl chloride monomer, especially an essentially metal-free activated carbon catalyst for catalytic conversion of ethylenedichloride (EDC) into vinyl chloride monomer (VCM).

### BACKGROUND

Vinyl chloride monomer (VCM) is used to produce polyvinyl chloride (PVC), one of the most popular engineering plastics widely used worldwide. There are two main pathways to manufacture VCM in the PVC industry: 1) the acetylene hydrochlorination reaction; and 2) thermal dehydrochlorination of ethylenedichloride (EDC) or 1,2-dichloroethane (1,2-DCE). The acetylene hydrochlorination reaction has recently attracted considerable attention in countries enriched with coal sources, however, faces the challenging bottleneck of huge energy consumption in the production of the raw material of calcium carbide and the serious environmental pollution caused by the highly toxic and volatile mercuric chloride catalyst used for acetylene hydrochlorination reaction. In contrast, the dehydrochlorination of EDC method is supposed to be cleaner and currently plays a major role in the VCM production from the oil-based ethylenic raw material. Conventionally, the thermal dehydrochlorination of EDC is performed at the temperature of 480-530° C. using 10-20 barg of pressure with the reaction expression shown in equation (1), generally provides an EDC conversion of about 50-60% and selectivity to VCM of about 99%. Unfortunately, the thermal dehydrochlorination suffers from the coking deposition in the tubular reactors. Hence, the industrial operation of EDC pyrolysis process is discontinuous, i.e., the unit has to be shut down for decoking process every certain intervals dependent on types of reactor, feedstocks, and operating conditions.

C₂H₄Cl₂(EDC)→C₂H₃Cl(VCM)+HCl ΔH=−19 kcal/g (1)

Conventionally, the 1,2-DCE pyrolysis process proceeds via free-radical reactions, and the coke deposition is resulted partially from the chloroethyl radicals. Therefore, the catalytic dehydrochlorination of EDC received extensively attention to explore a dehydrochlorination process at lower temperature to suppress the coke deposition. For instances, Eberly et al. disclosed in U.S. Pat. No. 2,875,255 of a ZnCl₂ supported catalyst with an EDC dehydrochlorination conversion of 67.3% at 475° C.

In 1966, Okamoto et al. reported the use of activated carbon catalyst for EDC dehydrochlorination, exhibiting an initial EDC conversion of 98.3% at high length temperature of 380° C. with total liquid hourly space velocity (LHSV) of 0.2 h⁻¹. In 1996, Sotowa et al. used the pyridine deposited pitch-based active carbon fiber as the EDC dehydrochlorination catalyst, achieving an initial EDC conversion about 60% at 360° C. and the LHSV of 0.5 h⁻¹, but the catalytic activity lost totally in 200 h owing to the pore blocking caused by coking. Similarly, in 1994, Mochida et al. used polyacrylonitrile-based active carbon fibers as the EDC dehydrochlorination catalyst, providing an initial EDC conversion about 50% at 360° C. and the LHSV of 1.7 h⁻¹, also similar to Sotowa, the activity lost totally in 50 h due to the coking. In 2014, Swietlik et al. reported nitrogen-doped ordered mesoporous carbon prepared from resorcinol and formaldehyde as the carbon precursor and dicyandiamide as the nitrogen precursor, showing the EDC conversion about 80% at 300° C. and 10 h reaction time.

Recently, CN 106732772 B discloses a mixed metal chloride and nitrogen-containing compounds active component formed on porous carrier for catalytically cracking 1,2-dichloroethane with conversion between 60-80% at 200-300° C. with over 120 days of service life. TW 1679060 B discloses a supported nitrogen-containing carbon catalyst for cracking 1,2-dichloroethane to produce vinyl chloride and a regeneration method. The catalyst supported on the surface of inorganic porous support, however, shows only 36-72% dichloroethane conversion at 240-260° C. Furthermore, CN 105833892 A discloses an active carbon carrier loaded with 16.7-44.4 mass percent of the nitrogen-containing compound having single-pass conversion rate of dichloroethane of up to 93% at the service life of 10 hours.

From these prior arts, apparently, person skilled in the arts must considered the benefits of high conversion and/or selectivity rates at the cost of the catalyst service life which generally far lower than 100 hours for carbon-based catalyst for conversion of 1,2-dichloroethane. Therefore, it is still a challenge to further explore for efficient carbon-based catalyst for EDC catalytic dehydrochlorination of with high activity but low coking deposition, possibly at low temperature, with long service hours.

The inventors unexpectedly found that N-doped activated carbon catalysts prepared using ammonia gas as the nitrogen precursor provides excellent performances for the catalytic dehydrochlorination of EDC comparable to the conventionally used metal-based catalytic reaction.

### SUMMARY

In one embodiment, the present disclosure provides an N-doped activated-carbon catalyst composition comprising a support having deposited thereon a catalytically active nitrogen, one or more alkaline or alkaline earth metals; wherein the support is carbon-based porous material.

In one aspect of the above embodiment of this invention, the N-doped activated-carbon catalyst comprising the content of catalytically active nitrogen from 1-7 wt% as characterized by elemental analysis.

In another aspect, the N-doped activated-carbon catalyst having the % nitrogen per surface area from 0.0005-0.006 g/m² as measured based on the N2 adsorption isotherm and calculated using the Brunauer-Emmett-Teller (BET) method.

In other aspect of the above embodiment of this invention, the carbon-based porous material having pore size distribution between 5-15 Å with deferential pore volume more than cc/g as measured using surface area analyser.

In further aspect of the embodiment, the N-doped activated-carbon catalyst having silicon content from 0.005-1 wt % as determined by wavelength-dispersive X-ray fluorescence (WDXRF) method. In still further aspect of this embodiment, the N-doped activated-carbon catalyst having sodium content from 0.0-0.2 wt % as determined by wavelength-dispersive X-ray fluorescence (WDXRF) method.

In one aspect, the N-doped activated-carbon catalyst of the present disclosure is prepared by treating activated-carbon with nitrogen precursor, such as ammonia gas, urea, ammonium hydroxide, or combination thereof. In one preferred aspect, the N-doped activated-carbon catalyst of the present disclosure is used in catalytic dehydrochlorination of EDC.

In one preferred aspect, the N-doped activated-carbon catalyst of this invention comprising 1-7% by weight of nitrogen and 1200-1800 m^{2 /}g of surface area.

In further aspect, the present disclosure provides an N-doped activated-carbon catalyst composition characterized in that the catalyst having the content of catalytically active nitrogen from 1-7 wt % as characterized by elemental analysis, the % nitrogen per surface area from 0.0005-0.006 g/m² as measured based on the N2 adsorption isotherm and calculated using the Brunauer-Emmett-Teller (BET) method, pore size distribution between 5-15 Å with deferential pore volume more than 0.1 cc/g as measured using surface area analyser, silicon content from 0.005-1 wt %, and sodium content from 0.0-0.2 wt % as determined by wavelength-dispersive X-ray fluorescence (WDXRF) method.

In one aspect, the N-doped activated-carbon catalyst of this invention exhibits a stable catalytic activity at 280° C. having an EDC conversion of 70-90% at more than 100 h.

In one aspect, the N-doped activated-carbon catalyst of this invention is used in the process for producing vinyl chloride monomer from EDC with the temperature between 250-300° C. and pressure between 5-20 barg.

In one preferred aspect, the N-doped activated-carbon catalyst of this invention is used in the process for producing vinyl chloride monomer from EDC with the temperature between 250-300° C. and pressure between 10-20 barg.

### BRIEF DESCRIPTION OF DRAWINGS

FIG. 1. Performance test for EDC catalytic cracking.

FIG. 2A. Pore size distribution of activated carbon IE1 and CE1 after ammonia treatment at 800-900° C., 2 h.

FIG. 2B. Pore size distribution of activated carbon IE2, IE3, CE2 and CE3 after ammonia treatment at 800-900° C., 2 h.

### DETAILED DESCRIPTION

Hereinafter, the present invention is now described in details in connection with the preferred embodiments. Various aspects of the present invention can be fully understood by way of examples.

Both synthetic and naturally sourced activated carbon were used. Various type of nitrogen precursor can be used, such as ammonia gas, urea, ammonium hydroxide, or combination thereof

### Example Catalyst Preparation

Synthetic activated carbon are used in IE1 (Inventive Example 1) and CE1 (Comparative Example 1) whereas activated carbon from natural sources are used in IE 2 (Inventive Example 2), IE 3 (Inventive Example 3), and CE2 (Comparative Example 2), CE3 (Comparative Example 3), respectively. The activated carbons with the particle size of 1.0-1.5 mm, are screened to prepare the N-doped catalyst. The reaction with ammonia is performed in a vertical tube furnace. The sample, packed in a quartz tube, is heated at 5 K/min with ammonia gas flow of 200 sccm (standard cubic centimeter per minute) to the final temperature (800° C. for CE1, 900° C. for IE1-3 and CE2-3, respectively) with 2 h soak.

### Catalyst Activity Evaluation

The evaluation of catalyst performance are performed using stainless steel tube (i.d. ¾ inch) fixed bed reactor. The pipeline is purged with nitrogen to remove water vapor and air in the system before each experiment. Liquid EDC, with the WHSV value of 3 h⁻¹, is fed into a vaporizing chamber by a liquid mass flow meter at a rate of 12 g/hour, where the EDC is vaporized to flow into the reactor with 4 g catalyst at a temperature of 280° C. under 10 bar by using back pressure control valve. The effluent of the reactor is condensed to separate the unreacted EDC and then passed into an absorption bottle containing 15% w/w NaOH solution (100 mL) to remove HCl, followed by the composition analysis using Agilent GC-3420A gas chromatograph with a hydrogen flame ionization detector (FID). The conversion of EDC is determined as disclosed by Z. shen et.al. React. Chem. Eng., 2018.

### Catalyst Characterization

The physical characterization of synthesized catalyst are conduct with surface area (SA) analyzer by N2 adsorption-desorption at −196° C. with quantachrome/Autosorb-1MP gas sorption system. To measurements, the sample is degassed at 300° C. for 12 h under vacuum. SA is calculated using the Brunauer-Emmett-Teller (BET) method from N2 adsorption isotherm. The pore size distributions are calculate by BJH method to show pore distributions peak between 5-15 Å, which suggests the promotion of catalyst activity with high pressure condition for EDC cracking to VCM product. The N content is determined by using ELEMENTAL ANALYZER—CHNS/O, LECO 628 Series. The Si and Na content are determined by using wavelength-dispersive X-ray fluorescence (WDXRF), S8 TIGER ECO.

TABLE 1
The effect of physical and chemical property on catalytic performance

| Sample | CE1 | IE1 | IE2 | IE3 | CE2 | CE3 |
|---|---|---|---|---|---|---|
| % Nitrogen | 7.36 | 3.28 | 2.93 | 4.15 | 1.22 | 1.34 |
| Surface area (SA)(m²/g) | 923.3 | 1792 | 1678.1 | 1773.5 | 1655.3 | 1650 |
| % N/SA (g/m²) | 0.00797 | 0.0018 | 0.0017 | 0.0023 | 0.00074 | 0.00081 |
| Si content | 59 ppm | 59 ppm | 0.05% | 0.08% | 1.10% | 1.31% |
| Wt % Na content | 0.06% | 0.06% | 0.16% | 0.10% | 0.05% | 0% |
| Conversion (TOS = 10 h) | 65 | 90 | 80 | 80 | 56 | 58 |
| % VCM selectivity | 99.7 | 99.6 | 99.8 | 99.8 | 98.6 | 99.3 |
| Pore size (5-15 Å) | No | Yes | Yes | Yes | Yes | Yes |
| TOS at conversion (<50%) | 20 | >100 | >100 | >100 | 20 | 22 |

Catalytic dehydrochlorination of EDC reaction is performed at 280° C. and WHSV (EDC)=3 h⁻¹over IE1, IE2, and IE3. The EDC conversion is shown in the region 70-95% with the selectivity of VCM equal to 99.5-99.8%. The stability of catalyst activity is more than 100 time on stream (TOS). The conversion and stability performance would be satisfied when the %Nitrogen in activated carbon is optimum amount as 1-7% by weight.

In addition, based on the concept of N atom dispersion in activated carbon would be promoted catalyst activity (Conversion>70% of EDC and stability>100 h of TOS), we have noted % Nitrogen per surface area (%N/SA) which situated within the optimum period of 0.0005-0.006 g/m². Whereas, otherwise, N-doped catalysts having EDC conversion significantly decreased, for instance, the EDC conversion is lower than 70% and low stability over CE1, CE2, and CE3 (%N/SA are 0.007971, 0.000737, and 0.000812, respectively).

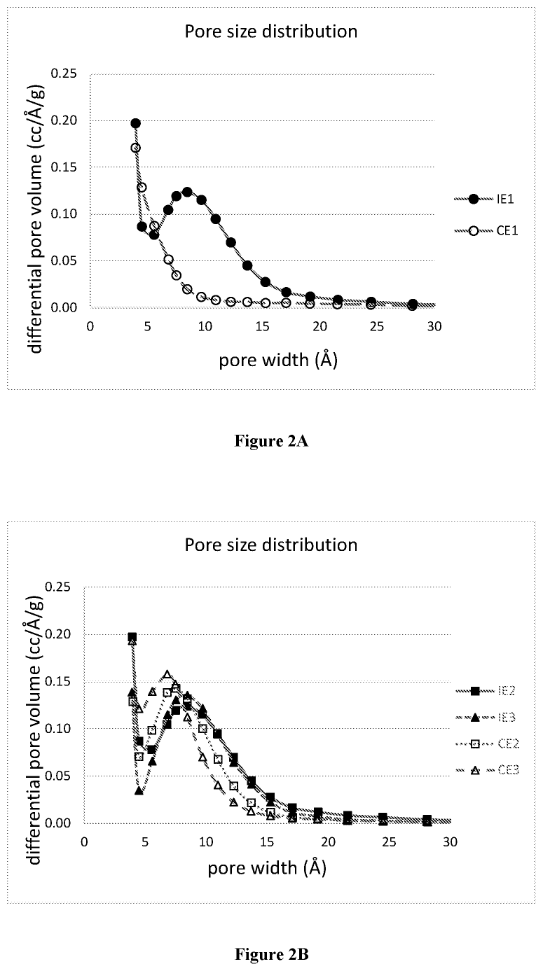

The pore size distribution of activated carbons after ammonia treatment with high temperature (800-900° C.) is shown in FIGS. 2A and 2B. It is found that higher treatment temperature causes the higher pore radius of activated carbon catalyst (IE1-3, and CE2-3, respectively). In other words, at temperature of 900° C., activated carbon porosity is more favorable developed by possibly enlarging the pore size and increasing surface area, while at the lower temperature the condition is not causing such development (CE1).

Furthermore, the activated carbon samples with high surface area, about 1000 m²/g are used for catalyst activity testing activated carbon in IE2-3, and CE2-3. The results of ammonia treatment at 900° C. are revealed in Table 1. The result shows agreeable correlation between the developed surface area and the catalyst activity.

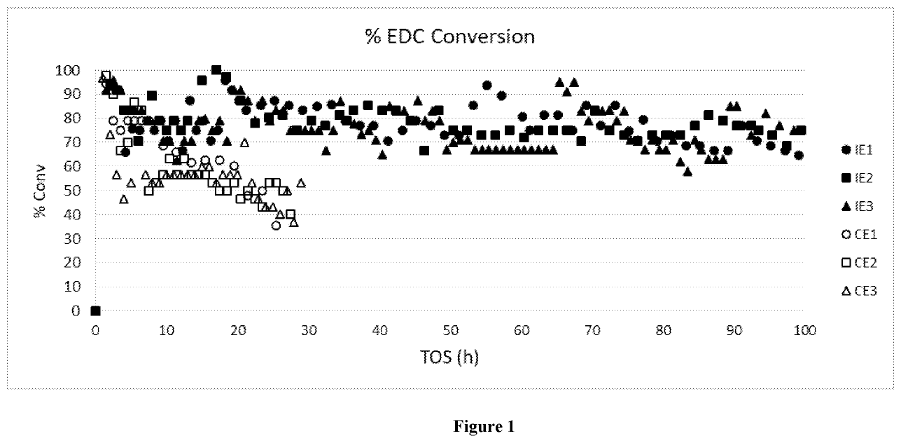

FIG. 2A and 2B shows the pore size distribution from different sources of activated carbon after ammonia treatment at 800-900° C. for 2 h. As indicated above, with the suitable pore width of activated carbon catalyst in the range of 5 -15 Å, all ammonia treated samples at 900° C. shown having pore size distribution in these range. For the performance of N-doped activated carbon requires not only pore width of 5-15 Å, but also requires optimum in %N/SA as shown in performance FIG. 1 and Table 1.

The results in FIGS. 2A and 2B showed that the optimum pore size of activated carbon after ammonia treatment should have the deferential pore volume more than 0.1 cc/kg in the range of 5-15 Å.

### The Effect of Impurity of Activated Carbon on VCM Selectivity

The XRF analysis of activated carbons supports our initial hypothesis that CE2, CE3 carbon sample are exhibited lower VCM selectivity than the others. It is found that the high Si content of CE2 and CE3 activated carbon sample could be the factor in lower than 99.5% of VCM selectivity.

1. An N-doped activated-carbon catalyst composition comprising a support having deposited thereon a catalytically active nitrogen, one or more alkaline or alkaline earth metals; wherein the support is carbon-based porous material.

2. The catalyst composition according to claim 1, wherein the N-doped activated-carbon catalyst comprising the catalytically active nitrogen from 1-7 wt % as characterized by elemental analysis.

3. The catalyst composition according to claim 1, wherein the N-doped activated-carbon catalyst having the % nitrogen per surface area from 0.0005-0.006 g/m² as measured based on the N2 adsorption isotherm and calculated using the Brunauer-Emmett-Teller (BET) method.

4. The catalyst composition according to claim 1, wherein the carbon-based porous material having pore size distribution between 5-15 Å with deferential pore volume more than 0.1 cc/g as measured using surface area analyser.

5. The catalyst composition according to claim 1, wherein the catalyst composition having silicon content from 0.005-1 wt % as determined by wavelength-dispersive X-ray fluorescence (WDXRF) method.

6. The catalyst composition according to claim 1, wherein the catalyst composition having sodium content from 0.0-0.2 wt % as determined by wavelength-dispersive X-ray fluorescence (WDXRF) method.

7. The catalyst composition according to claim 1, wherein the composition is prepared by treating activated-carbon with nitrogen precursor.

8. The catalyst composition according to claim 1, wherein the catalyst comprising 1-7% by weight of nitrogen and 1200-1800 m²/g of surface area.

9. The catalyst composition according to claim 1, wherein the catalyst composition is used in catalytic dehydrochlorination of ethylenedichloride (EDC).

10. The catalyst composition according to claim 1, wherein the catalyst composition exhibits a stable catalytic activity at 280° C. having an EDC conversion of 70-90% at more than 100 h.

11. A process for catalytic dehydrochlorination of EDC using the catalyst composition according to claim 1, wherein the catalyst composition is used in the process for producing vinyl chloride monomer from EDC with the temperature between 250-300° C. and pressure between 5-20 barg.

12. A process for catalytic dehydrochlorination of EDC using the catalyst composition according to claim 1, wherein the catalyst composition is used for producing vinyl chloride monomer from EDC with the temperature between 250-300° C. and pressure between 10-20 barg.
